(19) United States
(12) Patent Application Publication
(10) Pub. No.: US 2011/0094511 A1
(43) Pub. Date: Apr. 28, 2011

(54) Method and Apparatus for Ventilator Safety Enhancement

(76) Inventors: Jason Zachariah, Mission Viejo, CA (US); Jerry D. Wright, JR., Coconut Creek, FL (US)

(21) Appl. No.: 12/913,279

(22) Filed: Oct. 27, 2010

Related U.S. Application Data
(60) Provisional application No. 61/255,420, filed on Oct. 27, 2009.

(51) Int. Cl. A61M 16/08

(52) U.S. Cl. 128/204.18

(57) ABSTRACT

A device for protecting a ventilated patient from the adverse consequences of disconnection within a ventilator circuit can be used within ventilator circuits found in either the bedside vent environment or a surgical setting requiring anesthesia. In a bedside vent environment, this device secures joints in the circuit from the proximal end of the wye to the distal end of the in-line suction catheter tubing. In particular, between these two ends, there might be a series of joints created by gas measurement sensors, heated moisture exchangers, and treatment delivery adapters. The inventive device described herein is capable of securing all these joint combinations as well. In a surgical setting, the device secures joints in the circuit from the proximal end of the wye to the distal end of the “elbow” connected to the artificial airway.

### RELATED APPLICATIONS

This patent application is related to and claims priority to U.S. Provisional Application Ser. No 61/255,420, filed Oct. 27, 2009, similarly entitled Method And Apparatus For Ventilator Safety Enhancement, which is herein incorporated by reference in its entirety.

### TECHNICAL FIELD

This patent disclosure relates generally to medical paraphernalia and apparatus and, more particularly to a method and apparatus for securing a ventilator circuit against accidental mid-circuit disconnection.

### BACKGROUND

It is often necessary during medical procedures and interventions to assist a patient in attaining adequate oxygenation. This is especially true of patients who, because of a trauma, acute/chronic illness or an administered substance, are unable to breath on their own. It is also true for patients who are breathing, but who nonetheless must receive more oxygen-rich air in order to achieve appropriate oxygenation.

Traditionally, such ventilation assistance is provided by means of a ventilator circuit which is secured to a patient's artificial airway. An artificial airway is defined as either an endotracheal tube (ET tube) or tracheostomy tube (trach tube). An ET tube can be found positioned within the patient's mouth or nose while a trach tube can be found positioned within the patient's trachea. The ventilator circuit has a first opening adapted to fit to the distal end of the artificial airway to dispense a gas such as oxygen to the patient. The ventilator circuit also has at least one other opening attached to a ventilator for receiving oxygen from a gas supply source and providing ventilatory support.

While such ventilation is sometimes provided just as a safeguard, in many cases, a patient that is receiving such ventilation is in dire and actual need of the assistance. It is thus important to ensure that the gas supply to the patient continues essentially uninterrupted. Indeed, such a patient may be seriously impaired or even fatally compromised if the ventilator circuit is ruptured or interrupted for any substantial period of time.

For this reason, there have been various attempts in the art, with varying degrees of success, to ensure the integrity of the gas supply between the ventilator circuit and the artificial airway. Such measures include one or more straps to ensure that the ventilator circuit remains affixed to the patient's artificial airway, as well as one or more fixtures to ensure that the ventilator circuit remains attached to the ET tube or trach tube. Mechanisms known in the art include U.S. patent applications US 2003/0196666 and US 2007/0181131. However, all such mechanisms are helpless, to prevent a disruption at the mid-circuit point, i.e., the joints between the proximal and distal ends of the ventilator circuit

Thus, unfortunately, there are still occasional instances when a patient may have their oxygenation disrupted due to a break in the gas supply even though the one end of the ventilator circuit remains affixed to the patient's artificial airway and the opposite end remains firmly affixed to the ventilator. As such, there is a long-felt and unresolved need in the art to prevent mid-circuit disconnection in the ventilator circuit during mechanical ventilation.

Moreover, disconnection of the gas supply during ventilation use can also raise the risk of patient infection. In particular, it is known that the contamination of bedside equipment by the patient's environmental surfaces (including the bed or floor) can result in the transmission of pathogens that can lead to hospital-acquired infection (HAI). The inventors have observed that disconnection of the ventilator circuit can result in the openings of the ventilator tubing or other components of the circuit to touch potentially contaminated surfaces. The inventors have further observed that this type of cross contamination can result in microbial pathogens entering the internal surface of the ventilator circuit, which can then have access into the respiratory tract of the patient. Ultimately, these pathogens have the potential to colonize within the patient and lead to a HAI.

The foregoing Background section is a discussion of underlying concepts, not prior art. This section is constructed by the inventors based on their own knowledge and observations, and except where expressly indicated otherwise using the words “it is known” or “prior art,” this section does not catalog, list or discuss the prior art.

### SUMMARY

The disclosure describes, in one aspect, a device for protecting a ventilated patient from the adverse consequences of disconnection at a series of one or more joints in a ventilator circuit other than the point where the ventilator circuit meets an ET tube or trach tube, referred to herein as the artificial airway. The inventive device described herein can be used within ventilator circuits found in either the bedside vent environment or a surgical setting requiring anesthesia. In a bedside vent environment, this device secures joints in the circuit from the proximal end of the wye to the distal end of the in-line suction catheter tubing. In particular, between these two ends, there might be a series of joints created by gas measurement sensors, heated moisture exchangers, and treatment delivery adapters. The inventive device described herein is capable of securing all these joint combinations as well.

In a surgical setting, the device secures joints in the circuit from the proximal end of the wye to the distal end of the “elbow” connected to the ET tube. Between these two ends, there may be joints created by heated moisture exchangers and gas measurement sensors. This device is capable of securing all such joint combinations as well.

The inventive device described herein may be used for ventilator set-ups for humans (all ages including neonates, pediatric, and adult) as well as for animals. This application can be used in short-term and long-term acute care hospitals, outpatient surgery centers, and long term care facilities with respiratory care units, and with patients on ventilator support at home. In addition, the device is usable in veterinary hospitals and clinics.

Other features, objects, and advantages of the invention will be appreciated by those of skill in the art upon reading the following detailed description and included figures, of which:

### BRIEF DESCRIPTION OF THE DRAWINGS

FIG. 1 is a schematic side view of a device according to an embodiment of the invention adapted to protect a ventilated patient from adverse consequences of ventilator tubing disconnection;

FIG. 2 is a schematic side view of a ventilator circuit including tubing connected to the ventilator and going to the point of the artificial airway;

FIG. 3 is a schematic exploded view showing joints in the ventilator circuit in a bedside vent environment;

FIG. 4 is a schematic side view showing the apparatus according to an embodiment of the invention as used to secure multiple joints;

FIG. 5 is a schematic view of the device according to an embodiment of the invention adapted for use in a surgical setting from the proximal end of the wye to the distal end of the “elbow” connected to the ET tube, between which points, there are a series of joints created by heated moisture exchangers and gas measurement sensors; and

FIG. 6 is a schematic side view showing a series of interconnected joints within which environment the device according to an embodiment of the invention may be used to prevent disconnection;

FIG. 7 is a perspective view according to an alternative embodiment of the invention; and

FIG. 8 is a perspective side view of a modification employed to allow use of the described device for a patient having an endotracheal tube.

### DETAILED DESCRIPTION

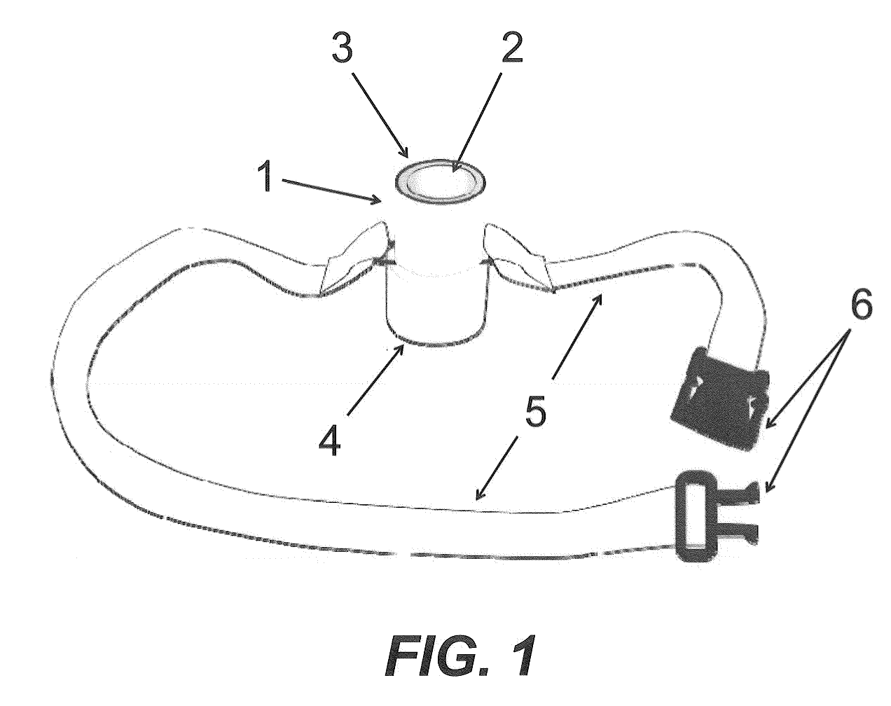

This disclosure relates to a system and apparatus for protecting a ventilated patient from the adverse consequences of ventilator tubing disconnection at a series of one or more joints in a ventilator circuit other than the point where the ventilator tubing meets an ET tube or trach tube. The device, shown in FIG. 1, is usable, in particular, to physically restrain the joints of interest from opening or separating. In greater detail, the device includes several components of interest including a joint tube 1 for insertion into the ventilation circuit, a first end 3 for joining to a first side of the circuit and a second distal end 4 for insertion into a further portion of the ventilation circuit, as will become more apparent by reference to later figures. The tube 1 is hollow, with the first 3 and second 4 ends being joined by an interior passage 2.

The device according to the illustrated embodiment of the invention also includes a plurality of rings or other retaining means on the tube 1, to each of which is affixed a strap portion 5. Each strap portion 5 in turn is terminated by a latch portion 6. It will be appreciated by those of skill in the art that the latch portions 6 are adapted to lockingly mate together, such that once joined, they may not be disconnected without first depressing a latch ramp or other lock mechanism. Thus, the latch portions 6 allow the two strap portions 5 to be joined in a way that they will not become unintentionally separated from one another.

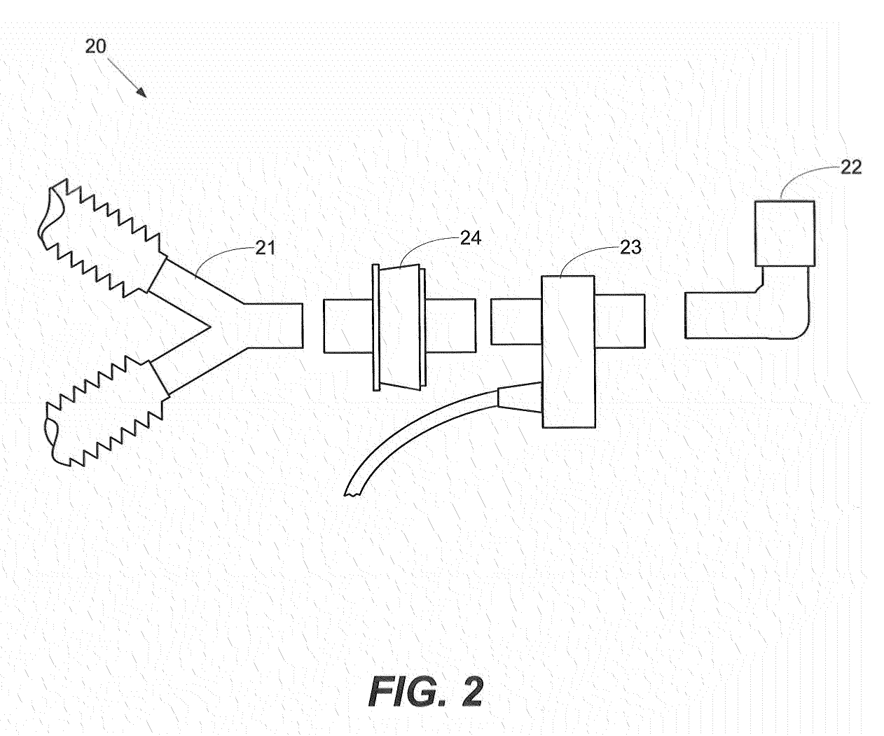

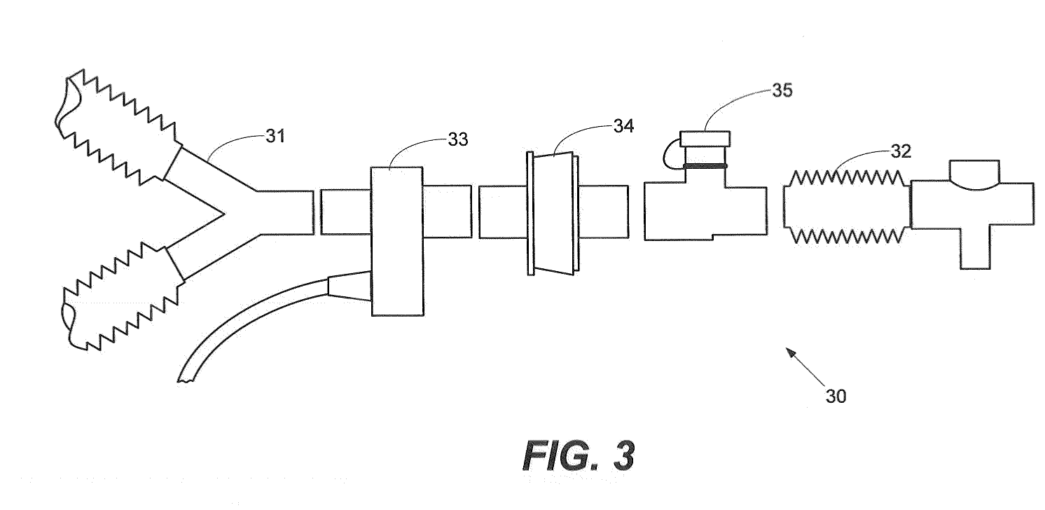

The ventilator circuit within which the device of FIG. 1 may be used is shown in greater detail with reference to FIG. 2. The circuit 20 includes a “wye” tube 21, an adapter 22 to the proximal end of the elbow (see FIG. 2). In between these two ends, there might be a series of, joints created by gas measurement sensors 23, heated moisture exchangers 24, and treatment delivery adapters (see FIG. 3).

As noted above embodiments of the invention can be used within ventilator circuits found in either the, bedside vent environment or a surgical setting requiring anesthesia. In a bedside vent environment 30, the inventive device secures joints in the circuit as shown in FIG. 3, from the proximal end of the wye 31 to the distal end of the in-line suction catheter tubing 32. In between these two ends, there is typically a series of joints created by gas measurement sensors 33, heated moisture exchangers 34, and treatment delivery adapters 35. This device according to embodiments of the invention is capable of securing all these joint combinations.

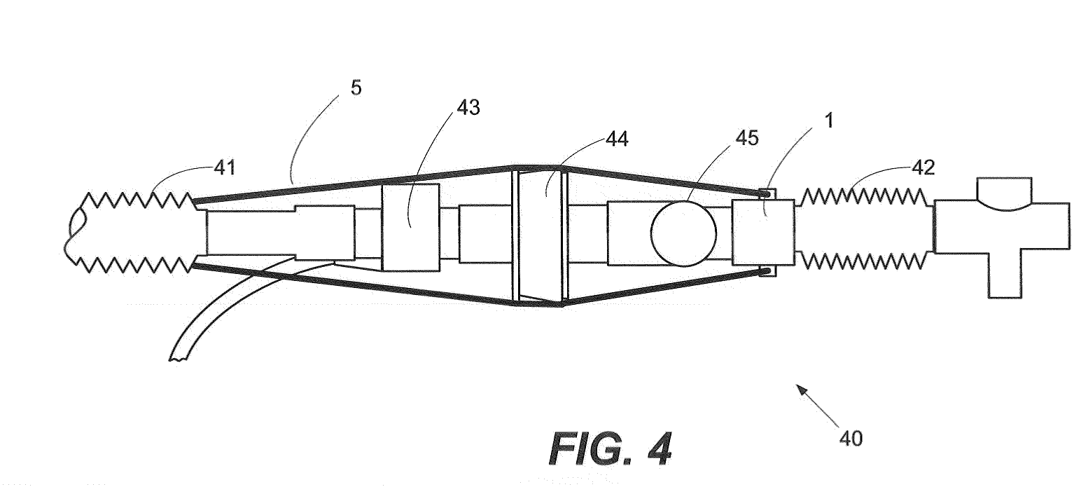

In FIG. 4, the device is shown applied to a circuit such as that of FIG. 3. As can be seen, the tube 1 of the device is installed after the treatment delivery adapter 45. The straps 5 of the device are passed through the opening of the wye 41 and secured via the latch portions 6 thereof. It will be appreciated that an adjustable slide piece, turnbuckle, or other tightening means (not shown) may also be incorporated on one or both straps 5. In practice, the device must be tightened such that any free lengths of strapping between the held ends will be essentially straight and tight, preventing any of the protected joints from opening.

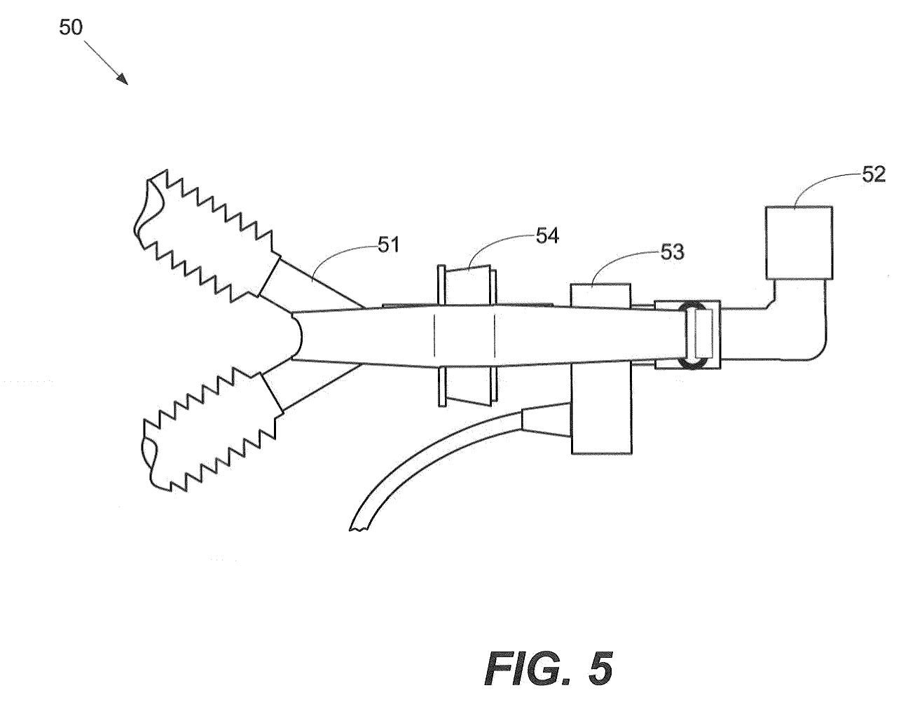

Referring now to FIG. 5, in a surgical setting, this device secures joints in the circuit from the proximal end of the wye 51 to the distal end of the elbow 52 connected to the ET tube. Between these two ends, there are typically several serial joints created by heated moisture exchangers 54 and gas measurement sensors 53. This device is capable of securing all these joint combinations as shown.

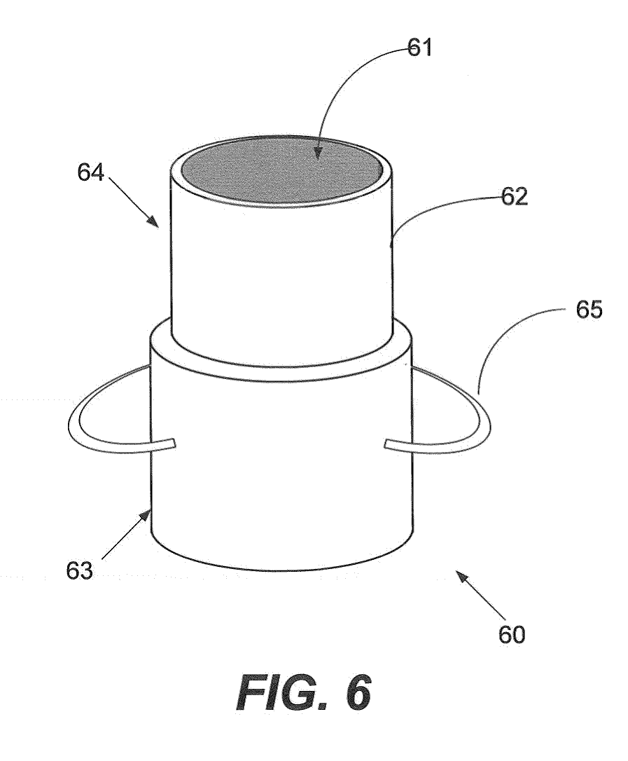

Turning finally to FIG. 6, this figure shows a perspective view of a safety device 60 in keeping with the disclosed principles. The device 60 as illustrated includes a hollow tube having an internal air passage 61 and an external surface 62, with a first end 63 of the tube being adapted to fit over a first mating tube portion and a second end 64 of the tube being adapted to fit within a second mating tube portion. In this manner, first and second tube portions that would otherwise mate to each other are joinable via the device 60. Two or more rings 65 are evenly spaced about the tube and are retained on the external surface 62 of the hollow tube. The device 60 is adapted to be used with one or more straps (not shown in this figure) via the rings 65, in conjunction with a latch portion attached to at least one of the straps so as to secure the device to a portion of the ventilation circuit, thereby preventing a mid-circuit disconnection.

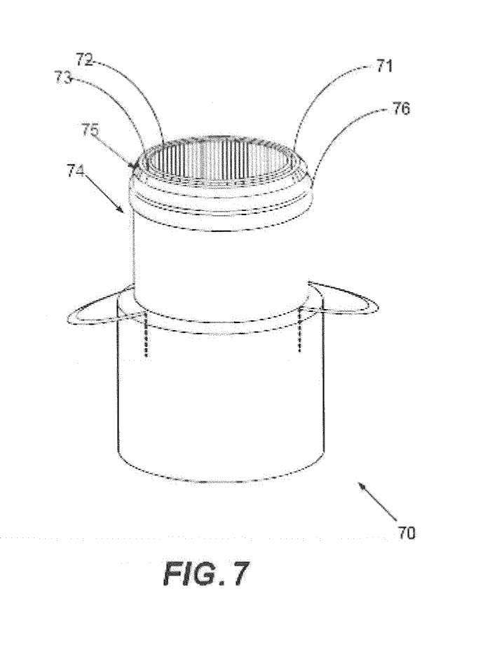

FIG. 7 shows a perspective view similar to that of FIG. 6, but according to a slightly different embodiment of the device. In particular, while the device 70 shares many of the same features as device 60, it differs at the second end 64 (74) of the device 60. The second end 74 of the device 70 includes a dual ring structure wherein an outer. ring 71 and an inner ring 72 are separated by a channel 73. In this way, the second end 74 is able to fit to different tube sizes, i.e., tubes that are sized to mate to any of: the outer surface of end 74, the inner surface of ring 71, the outer surface of ring 72, and the inner surface of ring 72. In addition, according to an embodiment usable with or without the additional rings 71, 72, the device 70 includes a bevel 75 on the outer surface of the second end 74. A lip 76 is also found on the outer surface of the second end 74. The bevel 75 and lip 76 aid in the smooth connection and locking of tubes onto the outer surface.

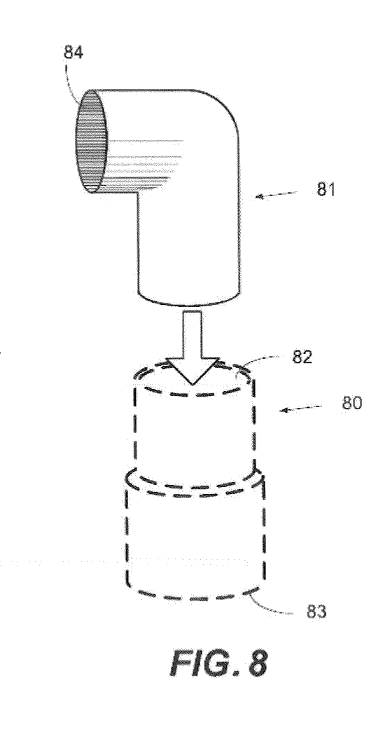

In an embodiment of the invention, the device 70 is usable in patients with endotracheal tubes or tracheostomy tubes requiring an elbow adapter. As shown in FIG. 8, the device 80 may be coupled to an elbow tube to allow such use. In the illustrated embodiment, the elbow tube 81 is inserted in the proximal end 82 of the device 80, i.e., opposite the distal end 83. The free end 84 of the elbow tube 81 is then joined with the endotracheal tube (not shown). In this way, the invention is also usable to prevent disconnection of the airway circuit in the context of an endotracheal tube as well as in other contexts.

It will be appreciated that a new and useful device has been provided and described for ensuring that a ventilated patient does not suffer from a mid-circuit disconnection. Use of this device should eliminate almost all mid-circuit disconnection events, accounting for 30% of all disconnection events. Moreover, as previously noted, the device according to embodiments of the invention can be used for ventilator set-ups for humans (all ages including neonates, pediatric, and adult) and animals. This application can be used in short-term and long-term acute care hospitals, outpatient surgery centers, long term care facilities with respiratory care units, and patients on ventilator support at home. In addition, it can be used in veterinary hospitals and clinics. Thus, this device has the potential to alleviate much needless suffering and injury.

It will be appreciated that the foregoing description provides examples of the disclosed system and apparatus. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A safety device for use in a patient ventilation circuit for preventing mid-circuit disconnection, the safety device comprising:
a hollow tube having an internal air passage and an external surface, with a first end of the tube being adapted to fit over a first mating tube portion and a second end of the tube being adapted to fit within a second mating tube portion, said first and second tube portions being shaped to otherwise mate to each other;
a plurality of rings evenly spaced and retained on the external surface of the hollow tube;
one or more straps having a first strap end terminating on a first one of the plurality of rings and a second strap end terminating on a second one of the plurality of rings; and
a latch portion attached to at least one of the one or more straps and being adapted to link the strap portions such that the hollow tube becomes secured to a portion of the ventilation circuit, thereby preventing a mid-circuit disconnection.

2. The device of claim I wherein the device is constructed of a material selected from the group consisting of silicon material and plastic material.

3. The device of claim 1 wherein the plurality of rings includes only two rings.

4. The device of claim 1 wherein the device is adapted for use in the medical arena to prevent mid-circuit disconnections.

5. The device of claim 1 wherein preventing a mid-circuit disconnection further comprises preventing accidental anoxic brain injury or death.

6. The device of claim I wherein preventing, a mid-circuit disconnection further comprises preventing hospital-acquired infections due to mid-circuit disconnection.

7. The device of claim 1 wherein the device is adapted for application in neonatal mechanical ventilation.

8. The device of claim 1 wherein the device is adapted for application in pediatric mechanical ventilation.

9. The device of claim 1 wherein the device is adapted for application in adult mechanical ventilation.

10. The device of claim 1 wherein the device is adapted for application in a hospital operating room for mechanical ventilation of patients undergoing general anesthesia.

11. The device of claim 1 where the device is adapted for application in one or more of Ambulatory Surgery Centers, Outpatient Surgery Centers, and Cosmetic Surgery Centers for mechanical ventilation of patients undergoing general anesthesia.

12. The device of claim 1 wherein the device is adapted for application in an intensive care units (ICU), coronary/cardiac care. unit (CCU), respiratory intensive care unit (RICU), special care unit (SCU) and post-anesthesia care unit (PACU) in short-term acute care hospitals whereby patients are Mechanically ventilated.

13. The device of claim 1 wherein the device is adapted for application in ICU, SCU, telemetry unit, or medical-surgical unit settings in long-term acute care hospitals whereby patients are mechanically ventilated.

14. The device of claim 1 wherein the device is adapted for application in a neonatal intensive care unit (NICU) or pediatric intensive care unit in short-term acute care hospitals whereby patients are mechanically ventilated.

15. The device of claim 1 wherein the device is adapted for application in long-term or chronic ventilator care unit settings in short-term acute care hospitals or skilled nursing facilities whereby patients are mechanically ventilated.

16. The device of claim 1 where the device is adapted for application in Veterinary Clinic or Veterinary Hospital settings for mechanically ventilated animals.

17. The device of claim 1 where the device is adapted for application in home care setting for mechanically ventilated patients.

18. The device of claim 1 where the device is adapted for application in hospice care setting for mechanically ventilated patients.
